(19) United States
(12) Patent Application Publication
(10) Pub. No.: US 2011/0082273 A1
(43) Pub. Date: Apr. 7, 2011

(54) HIGH-FUNCTIONAL POLYISOCYANATES CONTAINING ALLOPHANATE AND SILANE GROUPS

(75) Inventors: Hans-Josef Laas, Odenthal (DE); Thomas Baker, Koln (DE); Markus Mechtel, Bergisch Gladbach (DE)

(73) Assignee: Bayer MaterialScience AG, Leverkusen (DE)

(21) Appl. No.: 12/894,348

(22) Filed: Sep. 30, 2010

(30) Foreign Application Priority Data
Oct. 1, 2009 (DE) 102009047964.3

(51) Int. Cl. C08G 77/26; C07C 263/16

(52) U.S. Cl. 528/38; 560/336

(57) ABSTRACT

The invention relates to high-functional polyisocyanates containing allophanate and silane groups, a method for their production and their use as a starting component in the production of polyurethane plastics, in particular as a crosslinker component in polyurethane paints and coatings.

### RELATED APPLICATIONS

This application claims benefit to German Patent Application No. 10 2009 047 964.3, filed Oct. 1, 2009, which is incorporated herein by reference in its entirety for all useful purposes.

### BACKGROUND OF THE INVENTION

The invention relates to high-functional polyisocyanates containing allophanate and silane groups, a method for their production and their use as a starting component in the production of polyurethane plastics, in particular as a crosslinker component in polyurethane paints and coatings.

Polyisocyanate mixtures containing alkoxysilane groups have been known for a relatively long time. Such products which in addition to the isocyanate group contain a second reactive structure, i.e. one which is capable of crosslinking, were used in the past in various polyurethane systems and applications to achieve specific properties, for example to improve the adhesion, chemical or scratch resistance of coatings.

For example, WO 03/054049 describes isocyanate-functional silanes produced from low-monomer aliphatic or cycloaliphatic polyisocyanates and secondary aminopropyl trimethoxysilanes as adhesion promoters for polyurethane hot-melt adhesives.

According to the teaching of JP-A 2005015644 the adhesion of adhesives and sealants can be improved by using polyisocyanates or isocyanate prepolymers modified with N-substituted, i.e. secondary, aminopropyl alkoxysilanes.

EP-B 0 994 139 claims reaction products of aliphatic or cycloaliphatic polyisocyanates with deficit amounts of alkoxysilane-functional aspartic acid esters, such as are described in EP-A 0 596 360 as reaction partners for isocyanate-functional compounds, and optionally polyethylene oxide polyether alcohols as binders for one-component moisture-crosslinking coatings, adhesives or sealants having accelerated curing.

Reaction products of aliphatic or cycloaliphatic polyisocyanates with deficit amounts of alkoxysilane-functional aspartic acid esters or secondary aminoalkyl silanes are also described in WO 02/058569 as crosslinker components for two-component polyurethane primers.

EP-B 0 872 499 describes aqueous two-component polyurethane paints containing compounds having isocyanate and alkoxysilyl groups as the crosslinker component. The use of these special polyisocyanates leads to coatings having improved water resistance combined with high gloss.

Hydrophilically modified and thus more easily emulsifiable polyisocyanates containing alkoxysilane groups have likewise already been mentioned as crosslinker components for aqueous two-component paint and adhesive dispersions (e.g. EP-A 0 949 284).

In recent times reaction products of aliphatic and/or cycloaliphatic polyisocyanates with N,N-bis-(trialkoxysilylpropyl)amines have been proposed as a crosslinker component (EP-A 1 273 640) to improve the scratch resistance of solvent-containing heat-curing two-component PU automotive clear coats or top coats.

Common to all of these silane-group-containing polyisocyanate mixtures is that they are produced by partial reaction of unmodified polyisocyanates or polyisocyanate prepolymers with organofunctional silanes containing isocyanate-group-reactive groups, for example mercaptofunctional silanes, primary aminoalkylsilanes, secondary N-alkyl-substituted aminoalkylsilanes or alkoxysilane-functional aspartic acid esters.

Such a modification, however, inevitably leads to a lowering of the average isocyanate functionality relative to the starting polyisocyanates used, the effect of which intensifies as the desired silane content in the reaction product increases. In practice, however, polyisocyanate crosslinkers having as high an isocyanate functionality as possible are desired in the aforementioned applications, such as paints or adhesives for example, in order to achieve a high crosslink density.

Furthermore, as the degree of modification increases, the viscosity of the products rises dramatically too because of the thiourethane and in particular the urea groups introduced into the molecule, as a consequence of which these polyisocyanates containing silane groups can generally only be used with the use of considerable amounts of organic solvents in dissolved form.

The depicted disadvantages of silane-modified polyisocyanates with regard to low NCO functionalities and high viscosities can be circumvented very elegantly, however, by the method of EP-A 2 014 692. According to this method silane-group-containing hydroxyurethanes or hydroxyamides, which can be accessed from aminoalkylsilanes by means of a ring-opening reaction with cyclic carbonates or lactones, are reacted with excess amounts of monomeric diisocyanates to form stable, light-coloured allophanate polyisocyanates which even with high silane contents are characterised by high isocyanate functionalities combined with low viscosities.

The silane-group-containing allophanate polyisocyanates of EP-A 2 014 692 are suitable as crosslinker components for many different hydroxy- and/or amino-functional binders for the formulation of solvent-containing, solvent-free or aqueous polyurethane or polyurea systems.

High-solids two-component coating systems based on polyaspartic acid esters, such as are described in the applicant's previously unpublished patent application with filing number 102009016173.2, represent a particularly interesting application for silane-modified polyisocyanates. In particular, polyaspartate paints produced using the allophanate polyisocyanates described in EP-A 2 014 692 exhibit excellent direct adhesion on metallic substrates such as for example zinc, aluminium or cold-rolled steel, which conventionally can be coated only with difficulty, making it possible to dispense with a primer coat.

Although the silane-modified allophanate polyisocyanates obtainable by the method according to EP-A 2 014 692 already have comparatively high isocyanate functionalities, when combined with the only difunctional polyaspartic acid esters available today these are frequently not sufficient, however, to guarantee a sufficiently rapid surface drying of the coatings for practical applications. However, the possibility of dispensing with a primer coat only means a genuine reduction in painting times and hence an increase in productivity for the user of such coating agents if combined with correspondingly short drying times.

The object of the present invention was therefore to provide new silane-group-containing polyisocyanates which lead to much faster surface drying, even in combination with exclusively difunctional paint binders, and at the same time exhibit the excellent adhesion properties of the silane-modified allophanate polyisocyanates of the prior art.

This object was able to be achieved with the provision of the polyisocyanates modified according to the invention or the method for their production as described in more detail below.

### EMBODIMENTS OF THE INVENTION

An embodiment of the present invention is a method for producing polyisocyanates comprising allophanate groups comprising reacting
A) at least one silane-group-comprising hydroxyurethane and/or hydroxyamide obtained by the reaction of an aminosilane with a cyclic carbonate or lactone, and
B) at least one further polyvalent hydroxy-functional component having a molecular weight in the range of from 62 to 2000 g/mol,
with an amount in molar excess relative to the NCO-reactive groups of components A) and B) of
C) at least one diisocyanate having aliphatically, cycloaliphatically, araliphatically, and/or aromatically bonded isocyanate groups,
   and optionally subsequently removing the unreacted diisocyanate excess.

Another embodiment of the present invention is the above method, wherein component A) is the reaction product of an aminosilane of general formula (I)

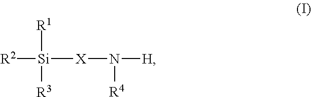

wherein
R¹, R², and R³ are, identically or differently, a saturated or unsaturated, linear or branched, aliphatic or cycloaliphatic radical having up to 18 carbon atoms or an optionally substituted aromatic or araliphatic radical having up to 18 carbon atoms, wherein said saturated or unsaturated, linear or branched, aliphatic or cycloaliphatic radical having up to 18 carbon atoms said optionally substituted aromatic or araliphatic radical can optionally comprise up to 3 heteroatoms selected from the group consisting of oxygen, sulfur, and nitrogen,
X is a linear or branched organic radical comprising at least 2 carbon atoms, which optionally comprise up to 2imino groups (—NH—), and
R⁴ is hydrogen, a saturated or unsaturated, linear or branched, aliphatic or cycloaliphatic radical having up to 18 carbon atoms, an optionally substituted aromatic or araliphatic radical having up to 18 carbon atoms, or a radical of formula

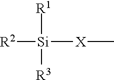

wherein R¹, R², R³, and X are as defined above,
with cyclic carbonates and/or lactones.

Another embodiment of the present invention is the above method, wherein component A) is the reaction product of an aminosilane of general formula (I)

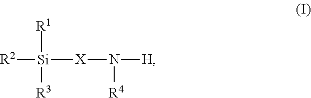

wherein
R¹, R², and R³ are, identically or differently, a saturated, linear or branched, aliphatic or cycloaliphatic radical having up to 6 carbon atoms, and which optionally comprises up to 3 oxygen atoms,
X is a linear or branched alkylene radical having from 2 to 10 carbon atoms, and which optionally comprises up to 2 imino groups (—NH—), and
R⁴ is hydrogen, a saturated, linear or branched, aliphatic or cycloaliphatic radical having up to 6 carbon atoms or a radical of formula

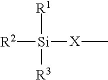

wherein R¹, R², R³, and X are as defined above,
with cyclic carbonates and/or lactones.

Another embodiment of the present invention is the above method, wherein component A) is the reaction product of aminosilane of general formula (I)

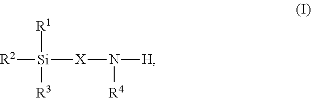

wherein
R¹, R² and R³ are each alkyl radicals having up to 6 carbon atoms and/or alkoxy radicals comprising up to 3 oxygen atoms, with the proviso that at least one of the radicals R¹, R², and R³ is an alkoxy radical,
X is a linear or branched alkylene radical having 3 or 4 carbon atoms, and
R⁴ is hydrogen, a methyl radical, or a radical of formula

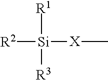

wherein R¹, R², R³, and X have the meaning given above,
   with cyclic carbonates and/or lactones.

Another embodiment of the present invention is the above method, wherein component A) is the reaction product of an aminosilane with ethylene carbonate, propylene carbonate, β-propiolactone, γ-butyrolactone, γ-valerolactone, γ-caprolactone, and/or ε-caprolactone.

Another embodiment of the present invention is the above method, wherein component B) is a polyhydric alcohol having a molecular weight in the range of from 62 to 400 g/mol and having 2 to 14 carbon atoms and/or an ester and/or an ether alcohol having a molecular weight in the range of from 106 to 400.

Another embodiment of the present invention is the above method, wherein component B) is a diol and/or triol having 2 to 6 carbon atoms.

Another embodiment of the present invention is the above method wherein the total amount of component B) is in the range of from 1 to 70 weight %, relative to the total amount of component A) used.

Another embodiment of the present invention is the above method, wherein component C) is a diisocyanate having aliphatically and/or cycloaliphatically bonded isocyanate groups.

Another embodiment of the present invention is the above method, wherein component C) is a 1,6-diisocyanatohexane, 1-isocyanato-3,3,5-trimethyl-5-isocyanatomethyl cyclohexane, 2,4′- and/or 4,4′-diisocyanatodicyclohexylmethane, or mixtures thereof.

Another embodiment of the present invention is the above method, wherein said reaction is performed in the presence of a catalyst which accelerates the formation of allophanate groups.

Another embodiment of the present invention is the above method, wherein said catalyst is a zinc compound and/or a zirconium compound.

Yet another embodiment of the present invention is an allophanate-group-containing polyisocyanate obtained by the above method.

Another embodiment of the present invention is the above allophanate-group-containing polyisocyanate, wherein said allophanate-group-containing polyisocyanate is blocked with blocking agents.

Yet another embodiment of the present invention is a polyurethane plastic prepared from the above allophanate-group-containing polyisocyanate.

Yet another embodiment of the present invention is a coating agent comprising the above allophanate-group-containing polyisocyanate.

Yet another embodiment of the present invention is a substrate coated with the above coating agent.

### DESCRIPTION OF THE INVENTION

The present invention is based on the surprising observation that silane-group-containing hydroxyurethanes or hydroxyamides, which are accessible by reacting aminoalkylsilanes with cyclic carbonates or lactones by means of a ring-opening reaction, can be reacted with excess amounts of monomeric diisocyanates and with incorporation of defined amounts of further diols and/or polyols to form high-functional allophanate polyisocyanates, which even with high silane contents have low viscosities and which lead to a clear reduction in drying times in comparison to the known silane-modified allophanate polyisocyanates combined with equally good metal adhesion.

The present invention provides a method for producing polyisocyanates containing allophanate groups by reacting
A) at least one silane-group-containing hydroxyurethane and/or hydroxyamide obtainable from the reaction of aminosilanes with cyclic carbonates or lactones and
B) at least one further polyvalent hydroxy-functional component having amolecular weight in the range of from 62 to 2000 g/mol
   with an amount in molar excess relative to the NCO-reactive groups of components A) and B) of
C) at least one diisocyanate having aliphatically, cycloaliphatically, araliphatically and/or aromatically bonded isocyanate groups
   and optionally subsequent removal of the unreacted diisocyanate excess.

The invention also provides the polyisocyanates containing allophanate and silane groups obtainable by this method and their use as starting components in the production of polyurethane plastics, in particular as a crosslinker component in polyurethane paints and coatings.

Starting compounds A) for the method according to the invention are any reaction products of aminosilanes with cyclic carbonates and/or lactones.

Suitable aminosilanes for producing the starting compounds A) are for example those of the general formula (I)

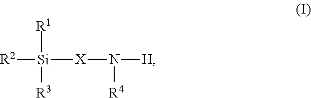

in which
R¹, R² and R³ stand for identical or different radicals and each denote a saturated or unsaturated, linear or branched, aliphatic or cycloaliphatic or an optionally substituted aromatic or araliphatic radical having up to 18 carbon atoms, which can optionally contain up to 3 heteroatoms from the oxygen, sulfur, nitrogen series,
X stands for a linear or branched organic radical having at least 2 carbon atoms, which can optionally contain up to 2 imino groups (—NH—), and
R⁴ stands for hydrogen, a saturated or unsaturated, linear or branched, aliphatic or cycloaliphatic or an optionally substituted aromatic or araliphatic radical having up to 18 carbon atoms or a radical of the formula

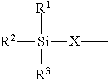

in which R¹, R², R³ and X have the meaning given above.

Suitable aminosilanes are, for example, 3-aminopropyl trimethoxysilane, 3-aminopropyl triethoxysilane, 3-aminopropyl methyl dimethoxysilane, 3-aminopropyl methyl diethoxysilane, 3-aminopropyl ethyl diethoxysilane, 3-aminopropyl dimethyl ethoxysilane, 3-aminopropyl diisopropyl ethoxysilane, 3-aminopropyl tripropoxysilane, 3-aminopropyl tributoxysilane, 3-aminopropyl phenyl diethoxysilane, 3-aminopropyl phenyl dimethoxysilane, 3-aminopropyl tris(methoxyethoxyethoxy)silane, 2-aminoisopropyl trimethoxysilane, 4-aminobutyl trimethoxysilane, 4-aminobutyl triethoxysilane, 4-aminobutyl methyl dimethoxysilane, 4-aminobutyl methyl diethoxysilane, 4-aminobutyl ethyl dimethoxysilane, 4-aminobutyl ethyl diethoxysilane, 4-aminobutyl dimethyl methoxysilane, 4-aminobutyl phenyl dimethoxysilane, 4-aminobutyl phenyl diethoxysilane, 4-amino(3-methylbutyl)methyl dimethoxysilane, 4-amino(3-methylbutyl)methyl diethoxysilane, 4-amino(3-methylbutyl)trimethoxysilane, 3-aminopropyl phenyl methyl-n-propoxysilane, 3-aminopropyl methyl dibutoxysilane, 3-aminopropyl diethyl methylsilane, 3-aminopropyl methyl bis(trimethylsiloxy)silane, 11-aminoundecyl trimethoxysilane, N-methyl-3-aminopropyl triethoxysilane, N-(n-butyl)-3-aminopropyl trimethoxysilane, N-(2-aminoethyl)-3-aminopropyl trimethoxysilane, N-(2-aminoethyl)-3-aminoisobutyl methyl dimethoxysilane, N-(2-aminoethyl)-3-aminopropyl methyl dimethoxysilane, N-(2-aminoethyl)-3-aminopropyl tris(2-ethylhexoxy)silane, N-(6-aminohexyl)-3-aminopropyl trimethoxysilane, N-benzyl-N-(2-aminoethyl)-3-aminopropyl trimethoxysilane, bis(3-trimethoxysilylpropyl)amine, bis(3-triethoxysilylpropyl)amine, (aminoethylaminomethyl)phenethyl trimethoxysilane, N-vinylbenzyl-N-(2-aminoethyl)-3-aminopropyl polysiloxane, N-vinylbenzyl-N-(2-aminoethyl)-3-aminopropyl polysiloxane, 3-ureidopropyl triethoxysilane, 3-(m-aminophenoxy)propyl trimethoxysilane, m- and/or p-aminophenyl trimethoxysilane, 3-(3-aminopropoxy)-3,3-dimethyl-1-propenyl trimethoxysilane, 3-aminopropyl methyl bis(trimethylsiloxy)silane, 3-aminopropyl tris(trimethylsiloxy)silane, 3-aminopropyl pentamethyl disiloxane or any mixtures of such aminosilanes.

Preferred aminosilanes for producing the starting component A) are those of the general formula (I), in which
R¹, R² and R³ stand for identical or different radicals and each denote a saturated, linear or branched, aliphatic or cycloaliphatic radical having up to 6 carbon atoms, which can optionally contain up to 3 oxygen atoms,
X stands for a linear or branched alkylene radical having 2 to 10 carbon atoms, which can optionally contain up to 2 imino groups (—NH—), and
R⁴ stands for hydrogen, a saturated, linear or branched, aliphatic or cycloaliphatic radical having up to 6 carbon atoms or a radical of the formula

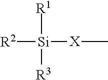

in which R¹, R², R³ and X have the meaning given above.

More preferred aminosilanes for producing the starting component A) are those of the general formula (I), in which
R¹, R² and R³ each denote alkyl radicals having up to 6 carbon atoms and/or alkoxy radicals containing up to 3 oxygen atoms, with the proviso that at least one of the radicals R¹, R² and R³ stands for such an alkoxy radical,
X stands for a linear or branched alkylene radical having 3 or 4 carbon atoms, and
R⁴ stands for hydrogen, a methyl radical or a radical of the formula

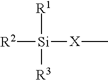

in which R¹, R², R³ and X have the meaning given above.

Particularly preferred aminosilanes for producing the starting component A) are those of the general formula (I), in which
R¹, R² and R³ each denote methyl, methoxy and/or ethoxy, with the proviso that at least one of the radicals R¹, R² and R³ stands for a methoxy or ethoxy radical,
X stands for a propylene radical (—CH₂—CH₂—CH₂—), and
R⁴ stands for hydrogen, a methyl radical or a radical of the formula

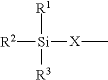

in which R¹, R², R³ and X have the meaning given above.

Most particularly preferred aminosilanes are aminopropyl trimethoxysilane, 3-aminopropyl triethoxysilane, 3-aminopropyl methyl dimethoxysilane and/or 3-aminopropyl methyl diethoxysilane.

In the production of the starting compounds A) for the method according to the invention the cited aminosilanes are reacted with any cyclic carbonates and/or lactones by means of a ring-opening reaction.

Suitable cyclic carbonates are in particular those having 3 or 4 carbon atoms in the ring, which can optionally also be substituted, such as for example 1,3-dioxolan-2-one (ethylene carbonate, EC), 4-chloro-1,3-dioxolan-2-one, 4,5-dichloro-1,3-dioxolan-2-one, 4-methyl-1,3-dioxolan-2-one (propylene carbonate, PC), 4-ethyl-1,3-dioxolan-2-one, 4,5-dimethyl-1,3-dioxolan-2-one, 4,4-dimethyl-1,3-dioxolan-2-one, 4-hydroxymethyl-1,3-dioxolan-2-one (glycerol carbonate), 4-phenoxymethyl-1,3-dioxolan-2-one, 1,3-dioxan-2-one (trimethylene carbonate), 5,5-dimethyl-1,3-dioxan-2-one, 5-methyl-5-propyl-1,3-dioxan-2-one, 5-ethyl-5-(hydroxymethyl)-1,3-dioxan-2-one (TMP carbonate), 4-isopropyl-5,5-dimethyl-1,3-dioxan-2-one (2,2,4-trimethylpentane-1,3-diol carbonate), 4-tert-butyl-5-methyl-1,3-dioxan-2-one (2,4,4-trimethylpentane-1,3-diol carbonate), 2,4-dioxaspiro[5.5]undecan-3-one (cyclohexane-1,1-dimethanol spirocarbonate) or any mixtures of such cyclic carbonates. Preferred cyclic carbonates are ethylene carbonate and/or propylene carbonate.

Suitable lactones are for example those having 3 to 6 carbon atoms in the ring, which can optionally also be substituted, such as for example β-propiolactone, β-butyrolactone, γ-butyrolactone, α-methyl-γ-butyrolactone, γ-valerolactone, γ-phenyl-γ-butyrolactone, α,α-diphenyl-γ-butyrolactone, γ-hexalactone (γ-caprolactone), γ-heptalactone, γ-octalactone, γ-nonalactone, γ-decalactone, γ-undecalactone, γ-dodecalactone, γ-methyl-γ-decanolactone, α-acetyl-γ-butyrolactone, δ-valerolactone, δ-hexanolactone, δ-octanolactone, δ-nonanolactone, δ-decalactone, δ-undecalactone, δ-tridecalactone, δ-tetradecalactone, γ-ethyl-γ-butyl-δ-valerolactone, octahydrocoumarin, ε-caprolactone, γ-phenyl-ε-caprolactone, ε-decalactone or any mixtures of such lactones. Preferred lactones are β-propiolactone, γ-butyrolactone, γ-valerolactone, γ-caprolactone and/or ε-caprolactone.

Production of the starting compounds A) by reacting the cited aminosilanes with the cyclic carbonates or lactones is known per se and can take place for example by the methods described in SU 295764, U.S. Pat. No. 4,104,296, EP-B 0 833 830 or WO 98/18844. As a general rule the reaction partners are reacted in equimolar amounts with one another at temperatures of 15 to 100° C., preferably 20 to 60° C. It is also possible, however, for one of the components, for example the aminosilane or the cyclic carbonate or lactone, to be used in an amount in molar excess, preferably however in an excess of at most 10 mol %, particularly preferably at most 5 mol %. The hydroxy-functional starting compounds A) obtainable in this way, which contain urethane groups if cyclic carbonates are used and amide groups if lactones are used, are generally colourless low-viscosity liquids.

In addition to the hydroxyurethanes or hydroxyamides A), at least one further polyvalent hydroxy-functional component B) in the molecular weight range from 62 to 2000 g/mol is used in the method according to the invention.

These are for example simple polyhydric alcohols having 2 to 14, preferably 2 to 6 carbon atoms, such as for example 1,2-ethanediol, 1,2- and 1,3-propanediol, the isomeric butanediols, pentanediols, hexanediols, heptanediols and octanediols, 1,10-decanediol, 1,2- and 1,4-cyclohexanediol, 1,4-cyclohexanedimethanol, 4,4′-(1-methylethylidene)-bis-cyclohexanol, 1,2,3-propanetriol, 1,1,1-trimethylolethane, 1,2,6-hexanetriol, 1,1,1-trimethylolpropane, 2,2-bis(hydroxymethyl)-1,3-propanediol, bis-(2-hydroxyethyl)hydroquinone, 1,2,4- and 1,3,5-trihydroxycyclohexane or 1,3,5-tris(2-hydroxyethyl)isocyanurate, but also simple ester or ether alcohols such as for example hydroxypivalic acid neopentyl glycol ester, diethylene glycol or dipropylene glycol.

Suitable hydroxy-functional components B) are also the higher-molecular-weight polyhydroxyl compounds known per se, of the polyester, polycarbonate, polyester carbonate or polyether type, in particular those in the molecular weight range from 200 to 2000 g/mol.

Polyester polyols which are suitable as hydroxy-functional components B) are for example those having an average molecular weight, calculable from the functionality and the hydroxyl value, of 200 to 2000 g/mol, preferably 250 to 1500 g/mol, with a hydroxyl-group content of 1 to 21 wt. %, preferably 2 to 18 wt. %, such as can be produced in a manner known per se by reacting polyhydric alcohols, for example those mentioned above having 2 to 14 carbon atoms, with deficit amounts of polybasic carboxylic acids, corresponding carboxylic anhydrides, corresponding polycarboxylic acid esters of low alcohols or lactones.

The acids or acid derivatives used to produce the polyester polyols can be of an aliphatic, cycloaliphatic and/or aromatic nature and optionally substituted, e.g. by halogen atoms, and/or unsaturated. Examples of suitable acids are for example polybasic carboxylic acids in the molecular weight range from 118 to 300 g/mol or derivatives thereof, such as for example succinic acid, adipic acid, sebacic acid, phthalic acid, isophthalic acid, trimellitic acid, phthalic anhydride, tetrahydrophthalic acid, maleic acid, maleic anhydride, dimeric and trimeric fatty acids, terephthalic acid dimethyl esters and terephthalic acid bis-glycol esters.

Any mixtures of these starting compounds cited by way of example can also be used to produce the polyester polyols.

A preferred type of polyester polyols for use as the hydroxy-functional component B) are those such as can be produced in a manner known per se from lactones and simple polyhydric alcohols, such as for example those cited above by way of example, as starter molecules by means of a ring-opening reaction. Suitable lactones for the production of these polyester polyols are for example β-propiolactone, γ-butyrolactone, γ- and δ-valerolactone, ε-caprolactone, 3,5,5- and 3,3,5-trimethylcaprolactone or any mixtures of such lactones.

Polyhydroxyl compounds of the polycarbonate type which are suitable as hydroxy-functional components B) are in particular the polycarbonate diols known per se, such as can be produced for example by reacting dihydric alcohols, for example those cited above by way of example in the list of polyhydric alcohols in the molecular weight range from 62 to 400 g/mol, with diaryl carbonates, such as for example diphenyl carbonate, dialkyl carbonates, such as for example dimethyl carbonate, or phosgene.

Polyhydroxyl compounds of the polyester carbonate type which are suitable as hydroxy-functional components B) are in particular the diols known per se having ester groups and carbonate groups, such as can be obtained for example according to the teaching of DE-A 1 770 245 or WO 03/002630 by reacting dihydric alcohols with lactones of the type cited above by way of example, in particular ε-caprolactone, and then reacting the polyester diols thus obtained with diphenyl carbonate or dimethyl carbonate.

Polyether polyols which are suitable as hydroxy-functional components B) are in particular those having an average molecular weight, calculable from the functionality and the hydroxyl value, of 200 to 2000 g/mol, preferably 250 to 1500 g/mol, with a hydroxyl-group content of 1.7 to 25 wt. %, preferably 2.2 to 20 wt. %, such as are accessible in a manner known per se by alkoxylation of suitable starter molecules. Any polyhydric alcohols can be used as starter molecules to produce these polyether polyols, such as the simple polyhydric alcohols described above having 2 to 14 carbon atoms. Suitable alkylene oxides for the alkoxylation reaction are in particular ethylene oxide and propylene oxide, which can be used in the alkoxylation reaction in any sequence or in a mixture.

Suitable polyether polyols are also the polyoxytetramethylene glycols known per se, such as can be obtained for example by polymerisation of tetrahydrofuran in accordance with Angew. Chem. 72, 927 (1960).

Preferred hydroxy-functional components B) for the method according to the invention are the aforementioned simple polyhydric alcohols in the molecular weight range from 62 to 400 g/mol and/or ester and/or ether alcohols in the molecular weight range from 106 to 400 g/mol.

The diols and/or triols having 2 to 6 carbon atoms cited above in the list of simple polyhydric alcohols are particularly preferred.

Most particularly preferred hydroxy-functional components B) are 1,2-ethanediol, 1,2-propanediol, 1,3-propanediol, 1,3-butanediol, 1,4-butanediol, 1,6-hexanediol and/or 1,1,1-trimethylolpropane.

The hydroxy-functional components B) are used in the method according to the invention in total in an amount of 1 to 70 wt. %, preferably 2 to 35 wt. %, particularly preferably 3 to 20 wt. %, relative to the total amount of hydroxyurethane and/or hydroxyamide A) used.

Any diisocyanates having aliphatically, cycloaliphatically, araliphatically and/or aromatically bonded isocyanate groups, which can be produced by any method, for example by phosgenation or by phosgene-free means, for example by urethane cleavage, are suitable as starting compounds C) for the method according to the invention. Suitable starting diisocyanates are for example those in the molecular weight range from 140 to 400 g/mol, such as for example 1,4-diisocyanatobutane, 1,6-diisocyanatohexane (HDI), 1,5-diisocyanato-2,2-dimethylpentane, 2,2,4- or 2,4,4-trimethyl-1,6-diisocyanatohexane, 1,10-diisocyanatodecane, 1,3- and 1,4-diisocyanatocyclohexane, 1,4-diisocyanato-3,3,5-trimethylcyclohexane, 1,3-diisocyanato-2-methylcyclohexane, 1,3-diisocyanato-4-methylcyclohexane, 1-isocyanato-3,3,5-trimethyl-5-isocyanatomethyl cyclohexane (isophorone diisocyanate; IPDI), 1-isocyanato-1-methyl-4(3)-isocyanatomethyl cyclohexane, 2,4′- and 4,4′-diisocyanatodicyclohexylmethane, 1,3- and 1,4-bis(isocyanatomethyl)cyclohexane, 4,4′-diisocyanato-3,3′-dimethyl dicyclohexylmethane, 4,4′-diisocyanato-3,3′,5,5′-tetramethyl dicyclohexylmethane, 4,4′-diisocyanato-1,1′-bi(cyclohexyl), 4,4′-diisocyanato-3,3′-dimethyl-1,1′-bi(cyclohexyl), 4,4′-diisocyanato-2,2′,5,5′-tetramethyl-1,1′-bi(cyclohexyl), 1,8-diisocyanato-p-menthane, 1,3-diisocyanatoadamantane, 1,3-dimethyl-5,7-diisocyanatoadamantane, 1,3- and 1,4-bis-(isocyanatomethyl)benzene, 1,3- and 1,4-bis(1-isocyanato-1-methylethyl)benzene (TMXDI), bis(4-(1-isocyanato-1-methylethyl)phenyl)carbonate, 1,3- and 1,4-phenylene diisocyanate, 2,4- and 2,6-toluylene diisocyanate and any mixtures of these isomers, diphenylmethane-2,4′- and/or -4,4′-diisocyanate and naphthylene-1,5-diisocyariate along with any mixtures of such diisocyanates. Further likewise suitable diisocyanates can be found moreover for example in Justus Liebigs Annalen der Chemie volume 562 (1949) p. 75-136.

The cited diisocyanates having aliphatically and/or cycloaliphatically bonded isocyanate groups are preferred as the starting component C).

Particularly preferred starting components C) for the method according to the invention are 1,6-diisocyanatohexane, 1-isocyanato-3,3,5-trimethyl-5-isocyanatomethyl cyclohexane, 2,4′- and/or 4,4′-diisocyanatodicyclohexylmethane or any mixtures of these diisocyanates.

To perform the method according to the invention the silane-group-containing hydroxyurethanes and/or hydroxyamides A) and at least one hydroxy-functional component B) are reacted with the diisocyanates C) at temperatures of 40 to 200° C., preferably 60 to 180° C., while maintaining an equivalents ratio of isocyanate groups to isocyanate-reactive groups of 4:1 to 50:1, preferably 5:1 to 30:1, to form allophanate polyisocyanates.

Within the meaning of the present invention “isocyanate-reactive groups” also include, in addition to the hydroxyl groups of components A) and B) and the urethane groups which form therefrom as intermediates due to NCO/OH reaction, the urethane groups already contained therein if hydroxyurethanes are used, since these likewise react further to allophanate groups under the reaction conditions.

The method according to the invention can be performed without catalysis as a thermally induced allophanatisation. However, suitable catalysts are preferably used to accelerate the allophanatisation reaction. These are the conventional known allophanatisation catalysts, for example metal carboxylates, metal chelates or tertiary amines of the type described in GB-A-0 994 890, alkylating agents of the type described in U.S. Pat. No. 3,769,318 or strong acids as described by way of example in EP-A-0 000 194.

Suitable allophanatisation catalysts are in particular zinc compounds, such as for example zinc(II) stearate, zinc(II) n-octanoate, zinc(II)-2-ethyl-1-hexanoate, zinc(II) naphthenate or zinc(II) acetylacetonate, tin compounds, such as for example tin(II) n-octanoate, tin(II)-2-ethyl-1-hexanoate, tin(II) laurate, dibutyl tin oxide, dibutyl tin dichloride, dibutyl tin diacetate, dibutyl tin dilaurate, dibutyl tin dimaleate or dioctyl tin diacetate, zirconium compounds, such as for example zirconium(IV)-2-ethyl-1-hexanoate, zirconium(IV) neodecanoate, zirconium(IV) naphthenate or zirconium(IV) acetylacetonate, aluminium tri(ethylacetoacetate), iron(III) chloride, potassium octoate, manganese, cobalt or nickel compounds and strong acids, such as for example trifluoroacetic acid, sulfuric acid, hydrogen chloride, hydrogen bromide, phosphoric acid or perchloric acid, or any mixtures of these catalysts.

Suitable albeit less preferred catalysts for the method according to the invention are also such compounds which in addition to the allophanatisation reaction also catalyse the trimerisation of isocyanate groups with formation of isocyanurate structures. Such catalysts are described for example in EP-A-0 649 866 page 4, line 7 to page 5, line 15.

Preferred catalysts for the method according to the invention are zinc and/or zirconium compounds of the aforementioned type. The use of zinc(II) n-octanoate, zinc(II)-2-ethyl-1-hexanoate and/or zinc(II) stearate, zirconium(IV) n-octanoate, zirconium(IV)-2-ethyl-1-hexanoate and/or zirconium(IV) neodecanoate is most particularly preferred.

The catalysts are used in the method according to the invention, if at all, in an amount from 0.001 to 5 wt. %, preferably 0.005 to 1 wt. %, relative to the total weight of the reaction partners A), B) and C), and can be added either before the start of the reaction or at any time during the reaction.

The method according to the invention is preferably performed without solvents. Optionally, however, suitable solvents which are inert in respect of the reactive groups of the starting components can be incorporated. Suitable solvents are for example the conventional paint solvents known per se, such as for example ethyl acetate, butyl acetate, ethylene glycol monomethyl or ethyl ether acetate, 1-methoxypropyl-2-acetate, 3-methoxy-n-butyl acetate, acetone, 2-butanone, 4-methyl-2-pentanone, cyclohexanone, toluene, xylene, chlorobenzene, white spirit, more highly substituted aromatics, such as are sold for example under the names solvent naphtha, Solvesso®, Isopar®, Nappar® (Deutsche EXXON CHEMICAL GmbH, Cologne, Del.) and Shellsol (Deutsche Shell Chemie GmbH, Eschborn, Del.), but also solvents such as propylene glycol diacetate, diethylene glycol dimethyl ether, dipropylene glycol dimethyl ether, diethylene glycol ethyl and butyl ether acetate, N-methyl pyrrolidone and N-methyl caprolactam, or any mixtures of such solvents.

In one possible embodiment, in the method according to the invention the starting diisocyanate C) or a mixture of various starting diisocyanates, optionally under inert gas, such as nitrogen for example, and optionally in the presence of a suitable solvent of the cited type, is set out at a temperature of between 20 and 100° C. Then the hydroxy-functional starting compounds A) and B) are added one after the other in any sequence or in a mixture in the amount specified above and the reaction temperature for urethanisation is optionally adjusted to a temperature of 30 to 120° C., preferably 50 to 100° C., by means of a suitable action (heating or cooling). Following the urethanisation reaction, i.e. when the NCO content theoretically corresponding to a complete conversion of isocyanate and hydroxyl groups is reached, allophanatisation can be initiated without addition of a catalyst for example by heating the reaction mixture to a temperature of 140 to 200° C. Suitable catalysts of the aforementioned type are preferably used, however, to accelerate the allophanatisation reaction, temperatures in the range from 60 to 140° C., preferably 80 to 120° C., being adequate, depending on the type and amount of catalyst used.

In another possible embodiment of the method according to the invention the catalyst which is optionally incorporated is added to either the silane component A), the hydroxy-functional component B) and/or the diisocyanate component C) before the start of the actual reaction. In this case the urethane groups which form as intermediates and which if hydroxyurethanes A) are used are already included therein, spontaneously react further to form the desired allophanate structure. In this type of single-stage reaction the starting diisocyanates C) optionally containing the catalyst are set out, optionally under inert gas, such as nitrogen for example, and optionally in the presence of a suitable solvent of the cited type, generally at optimum temperatures for allophanatisation in the range from 60 to 140° C., preferably 80 to 120° C., and reacted with the hydroxy-functional components A) and B) optionally containing the catalyst.

It is however also possible to add the catalyst to the reaction mixture at any point during the urethanisation reaction. In this embodiment of the method according to the invention a temperature generally in the range from 30 to 120° C., preferably 50 to 100° C., is set for the pure urethanisation reaction which takes place before the catalyst addition. After adding a suitable catalyst the allophanatisation reaction is finally performed at temperatures from 60 to 140° C., preferably 80 to 120° C.

The progression of the reaction can be monitored in the method according to the invention by for example determining the NCO content by titrimetry. The reaction is terminated after the desired NCO content has been reached, preferably when the degree of allophanatisation (i.e. the percentage of the urethane groups which form as intermediates from the hydroxyl groups of component A) and B) and which if hydroxyurethanes A) are used are already contained therein, that has been converted to allophanate groups, calculable from the NCO content) of the reaction mixture is at least 80%, particularly preferably at least 90%, most particularly preferably after complete allophanatisation. With a purely thermal reaction control this can be done for example by cooling the reaction mixture to room temperature. With the preferred incorporation of an allophanatisation catalyst of the cited type the reaction is however generally terminated by the addition of suitable catalyst poisons, for example acids, such as phosphoric acid, or acid chlorides, such as benzoyl chloride or isophthaloyl dichloride.

The reaction mixture is then preferably freed from volatile constituents (excess monomeric diisocyanates, cyclic carbonates or lactones optionally used in excess in the production of the starting compounds A), solvents optionally used and, if a catalyst poison is not used, optionally active catalyst) by film distillation under high vacuum, for example under a pressure of below 1.0 mbar, preferably below 0.5 mbar, particularly preferably below 0.2 mbar, under as gentle conditions as possible, for example at a temperature of 100 to 200° C., preferably 120 to 180° C.

The accumulating distillates, which in addition to the unreacted monomeric starting diisocyanates contain cyclic carbonates or lactones optionally used in excess and solvents optionally used and, if a catalyst poison is not used, optionally active catalyst, can be used for oligomerisation again without difficulty.

In a further embodiment of the method according to the invention, the cited volatile constituents are separated from the oligomerisation product by extraction with suitable solvents which are inert in respect of isocyanate groups, for example aliphatic or cycloaliphatic hydrocarbons such as pentane, hexane, heptane, cyclopentane or cyclohexane.

Regardless of the type of processing, clear, virtually colourless polyisocyanates are obtained as products of the method according to the invention, which have colour values of less than 200 APHA, preferably less than 100 APHA, particularly preferably less than 80 APHA, an average NCO functionality of 2.4 to 6.0, preferably 2.6 to 5.0, particularly preferably 3.2 to 4.8, and an NCO content of 6.0 to 21.0 wt. %, preferably 10.0 to 19.0 wt. %, particularly preferably 12.0 to 18.0 wt. %.

The allophanate polyisocyanates according to the invention are valuable starting materials for the production of polyurethane plastics by the isocyanate polyaddition method.

Owing to their comparatively low viscosity they can be used without solvent but can if necessary also be diluted with conventional solvents, for example the aforementioned inert paint solvents for optional use in the method according to the invention, without becoming cloudy.

The silane-modified allophanate polyisocyanates obtainable according to the invention are outstandingly suitable as hardeners for two-component polyurethane paints in which the conventional polyether polyols, polyester polyols, polycarbonate polyols and/or polyacrylate polyols are present as polyhydroxyl compounds as reaction partners for the polyisocyanates. Particularly preferred hydroxy-functional reaction partners for the process products according to the invention are polyacrylates having hydroxyl groups, i.e. polymers or copolymers of (meth)acrylic acid alkyl esters, optionally with styrene or other copolymerisable olefinically unsaturated monomers.

The process products according to the invention are also most particularly suitable as hardener components for amino-functional binders, in particular as crosslinkers in high-solids two-component coating systems based on polyaspartic acid esters, such as are described for example in the applicant's previously unpublished German patent application with filing number 102009016173.2.

Polyamines whose amino groups are in blocked form, such as for example polyketimines, polyaldimines or oxazolones, are also suitable reaction partners for the process products according to the invention. Under the influence of moisture such blocked polyamines re-form free amino groups and in the case of oxazolones also free hydroxyl groups, which are then available for crosslinking with isocyanate groups.

Coatings produced with the polyisocyanates containing silane groups according to the invention have exceptionally good adhesion to critical metallic surfaces and thus allow a direct application onto unprimed materials. In comparison to the allophanate polyisocyanates of EP-A 2 014 692 they have markedly improved drying characteristics, in particular also in combination with reaction partners having a low functionality, such as for example the difunctional polyaspartic acid esters of the type known from EP-B 0 403 921.

The coating agents formulated with the silane-modified allophanate polyisocyanates according to the invention, into which the auxiliary agents and additives conventionally used in the paint sector, such as for example flow control agents, coloured pigments, fillers or matting agents, can optionally be incorporated, generally have good paint properties even when dried at room temperature. Of course they can also be dried under forced conditions at elevated temperature or by stoving at temperatures of up to 260° C., however.

Suitable catalysts can be incorporated into the formulation of the coating agents to control the curing rate, for example the catalysts conventionally used in isocyanate chemistry, such as for example tertiary amines such as triethylamine, pyridine, methyl pyridine, benzyl dimethylamine, N,N-endoethylene piperazine, N-methyl piperidine, pentamethyl diethylene triamine, N,N-dimethyl aminocyclohexane, N,N′-dimethyl piperazine or metal salts such as iron(III) chloride, zinc chloride, zinc-2-ethyl caproate, tin(II) octanoate, tin(II) ethyl caproate, dibutyl tin(IV) dilaurate, bismuth(III)-2-ethyl hexanoate, bismuth(III) octoate or molybdenum glycolate. In addition, catalysts which accelerate the hydrolysis and condensation of alkoxysilane groups or their reaction with the hydroxyl groups of the polyol components used as binders can also be incorporated. In addition to the aforementioned isocyanate catalysts, such catalysts are also for example acids, such as for example p-toluenesulfonic acid, trifluoromethane sulfonic acid, acetic acid, trifluoroacetic acid and dibutyl phosphate, bases, such as for example N-substituted amidines such as 1,5-diazabicyclo[4.3.0]non-5-ene (DBN) and 1,5-diazabicyclo[5.4.0]undec-7-ene (DBU), but also metal salts or organometallic compounds, such as for example tetraisopropyl titanate, tetrabutyl titanate, titanium(IV) acetyl acetonate, aluminium acetyl acetonate, aluminium triflate or tin triflate.

The silane-modified allophanate polyisocyanates according to the invention can of course also be used in a form blocked with blocking agents known per se from polyurethane chemistry in combination with the aforementioned paint binders or paint binder components within the meaning of one-component PU stoving systems. Suitable blocking agents are for example malonic acid diethyl esters, acetoacetic esters, activated cyclic ketones, such as for example cyclopentanone-2-carboxymethyl ester and -carboxyethyl ester, acetonoxime, butanonoxime, ε-caprolactam, 3,5-dimethyl pyrazole, 1,2,4-triazole, dimethyl-1,2,4-triazole, imidazole, benzyl tert-butylamine or any mixtures of these blocking agents.

The invention therefore also provides the use of the polyisocyanates containing allophanate groups according to the invention for the production of polyisocyanates blocked with blocking agents known from polyurethane chemistry and the resulting blocked polyisocyanates themselves.

The silane-modified allophanate polyisocyanates according to the invention are also suitable as crosslinker components for binders or binder components dissolved or dispersed in water having isocyanate-group-reactive groups, in particular alcoholic hydroxyl groups, in the production of aqueous two-component polyurethane systems. Owing to their low viscosity they can either be used as such, i.e. in hydrophobic form, or also in hydrophilically modified form in accordance with known methods, for example in accordance with EP-B 0 540 985, EP-B 0 959 087 or EP-B 1 287 052.

Any further hydrolysable silane compounds, such as for example tetramethoxysilane, tetraethoxysilane, methyl trimethoxysilane, methyl triethoxysilane, ethyl triethoxysilane, isobutyl trimethoxysilane, isobutyl triethoxysilane, octyl triethoxysilane, octyl trimethoxysilane, (3-glycidyloxypropyl)methyl diethoxysilane, (3-glycidyloxypropyl)trimethoxysilane, phenyl trimethoxysilane or phenyl triethoxysilane, or mixtures of such silane compounds, can optionally be added as reaction partners to the coating systems formulated with the silane-modified allophanate polyisocyanates according to the invention.

In all paint combinations the process products according to the invention and their reaction partners are present in amounts such that 0.5 to 3, preferably 0.6 to 2.0, particularly preferably 0.8 to 1.6 optionally blocked, isocyanate-reactive groups are allotted to each optionally blocked isocyanate group.

The silane-modified allophanate polyisocyanates according to the invention can also be added in small amounts to non-functional paint binders, however, to achieve very specific properties, for example as additives to improve adhesion.

Any substrates are suitable as substrate materials for the coatings formulated with the aid of the silane-modified allophanate polyisocyanates according to the invention, such as for example metal, wood, glass, stone, ceramic materials, concrete, rigid and flexible plastics, textiles, leather and paper, which notwithstanding their good direct adhesion to a large number of materials can also be treated with conventional primers prior to coating.

This invention therefore also provides coating agents containing the polyisocyanates bearing allophanate groups according to the invention and substrates coated with these coating agents.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

Unless otherwise specified, all percentages are based on weight.

The NCO contents were determined in accordance with DIN EN ISO 11909.

All viscosity measurements were performed using a Physica MCR 51 rheometer from Anton Paar Germany GmbH (Ostfildern) in accordance with DIN EN ISO 3219.

The Hazen colour values were determined using a LICO 400 colour measuring instrument from Hach Lange GmbH, Düsseldorf.

The OH values given in the case of the starting compounds A) were calculated from the theoretically resulting molecular weight of the ideal structure (1:1 adduct).

Production of the Starting Compounds A)

Silane-Group-Containing Hydroxyurethane A1)

221 g (1.0 mol) of 3-aminopropyl triethoxysilane were set out at room temperature under dry nitrogen. 88 g (1.0 mol) of ethylene carbonate were added within 15 minutes whilst stirring, during which time the mixture heated up initially to 34° C. because of the heat of reaction being released, and the mixture was then stirred for 18 hours at room temperature with no further heating. An amine titration with 1N HCl showed a conversion of 99.8%.

2-Hydroxyethyl[3-(triethoxysilyl)propyl]urethane was obtained as a colourless liquid.

| | | |
|---|---|---|
| Viscosity (23° C.): | 69 | mPas |
| OH value (calc.): | 181 | mg KOH/g |
| Molecular weight (calc.): | 309 | g/mol |

Silane-Group-Containing Hydroxyurethane A2)

179 g (1.0 mol) of 3-aminopropyl trimethoxysilane and 88 g (1.0 mol) of ethylene carbonate were reacted together by the method described for starting compound A1). The conversion (amine titration with 1N HCl) after 18 hours was 99.6%.

2-Hydroxyethyl[3-(trimethoxysilyl)propyl]urethane was obtained as a colourless liquid.

| | | |
|---|---|---|
| Viscosity (23° C.): | 245 | mPas |
| OH value (calc.): | 210 | mg KOH/g |
| Molecular weight (calc.): | 267 | g/mol |

Silane-Group-Containing Hydroxyurethane A3)

221 g (1.0 mol) of 3-aminopropyl triethoxysilane and 102 g (1.0 mol) of propylene carbonate were reacted together by the method described for starting compound A1). The conversion (amine titration with 1N HCl) after 18 hours was 99.9%.

A mixture of 2-hydroxypropyl[3-(triethoxysilyl)propyl]urethane and 2-hydroxy-1-methylethyl[3-(triethoxysilyl)propyl]urethane was obtained as a colourless liquid.

| | | |
|---|---|---|
| Viscosity (23° C.): | 86 | mPas |
| OH value (calc.): | 173 | mg KOH/g |
| Molecular weight (calc.): | 323 | g/mol |

Silane-Group-Containing Hydroxyamide A4)

221 g (1.0 mol) of 3-aminopropyl triethoxysilane and 86 g (1.0 mol) of γ-butyrolactone were reacted together by the method described for starting compound A1). The conversion (amine titration with 1N HCl) after 18 hours was 99.4%.

4-Hydroxy-N[3-(triethoxysilyl)propyl]butanamide was obtained as a colourless liquid.

| | | |
|---|---|---|
| Viscosity (23° C.): | 326 | mPas |
| OH value (calc.): | 199 | mg KOH/g |
| Molecular weight (calc.): | 281 | g/mol |

### Example 1 (According to the Invention)

216.3 g (0.7 mol) of the silane-group-containing hydroxyurethane A1) and 18.6 g (0.3 mol) of 1,2-ethanediol, corresponding to an amount of 8.6 wt. % relative to the amount of hydroxyurethane Al), were added to 2520.0 g (15.0 mol) of hexamethylene diisocyanate (HDI) at a temperature of 80° C. under dry nitrogen and stirred for 3 hours until an NCO content of 43.8%, corresponding to complete urethanisation, was achieved. Then the reaction mixture was heated to 95° C. and 0.5 g of zinc(II)-2-ethyl-1-hexanoate were added as allophanatisation catalyst. Owing to the exothermic start to the reaction the temperature of the mixture rose to 110° C. After approx. 30 min the NCO content of the reaction mixture was 40.7%. The catalyst was deactivated by adding 1 g of benzoyl chloride and the unreacted monomeric HDI was separated off in a film evaporator at a temperature of 130° C. and under a pressure of 0.1 mbar. 697 g of a virtually colourless, clear allophanate polyisocyanate with the following characteristics were obtained:

| | |
|---|---|
| NCO content: | 16.2% |
| Monomeric HDI: | 0.04% |
| Viscosity (23° C.): | 1180 mPas |
| Colour value (APHA): | 20 Hazen |
| NCO functionality: | >3.3 (calculated) |
| Silane group content: | 8.1% (calculated as SiO₃; |
| | mol. weight = 76 g/mol) |

### Example 2 (According to the Invention)

1680.0 g (10.0 mol) of HDI were reacted with 247.2 g (0.8 mol) of the silane-group-containing hydroxyurethane A1) and 15.2 g (0.2 mol) of 1,3-propanediol, corresponding to an amount of 6.1 wt. % relative to the amount of hydroxyurethane A1), by the method described in Example 1. The allophanatisation reaction was started at an NCO content of 40.7% by the addition of 0.5 g of zinc(II)-2-ethyl-1-hexanoate. After reaching an NCO content of 36.3% the reaction mixture was stopped with 1 g of benzoyl chloride and processed as described in Example 1. 673 g of a virtually colourless, clear allophanate polyisocyanate with the following characteristics were obtained:

| | |
|---|---|
| NCO content: | 14.9% |
| Monomeric HDI: | 0.07% |
| Viscosity (23° C.): | 1550 mPas |
| Colour value (APHA): | 21 Hazen |
| NCO functionality: | >3.2 (calculated) |
| Silane group content: | 8.7% (calculated as SiO₃; |
| | mol. weight = 76 g/mol) |

### Example 3 (According to the Invention)

2520.0 g (15.0 mol) of HDI were reacted with 200.9 g (0.65 mol) of the silane-group-containing hydroxyurethane A1) and 31.5 g (0.35 mol) of 1,3-butanediol, corresponding to an amount of 15.7 wt. % relative to the amount of hydroxyurethane A1), by the method described in Example 1. The allophanatisation reaction was started at an NCO content of 43.7% by the addition of 0.5 g of zinc(II)-2-ethyl-1-hexanoate. After reaching an NCO content of 40.7% the reaction mixture was stopped with 1 g of benzoyl chloride and processed as described in Example 1. 631 g of a virtually colourless, clear allophanate polyisocyanate with the following characteristics were obtained:

| | |
|---|---|
| NCO content: | 16.1% |
| Monomeric HDI: | 0.08% |
| Viscosity (23° C.): | 1250 mPas |
| Colour value (APHA): | 19 Hazen |
| NCO functionality: | >3.4 (calculated) |
| Silane group content: | 6.9% (calculated as SiO₃; |
| | mol. weight = 76 g/mol) |

### Example 4 (According to the Invention)

2520.0 g (15.0 mol) of HDI were reacted with 278.1 g (0.9 mol) of the silane-group-containing hydroxyurethane A1) and 13.4 g (0.1 mol) of trimethylolpropane, corresponding to an amount of 4.8 wt. % relative to the amount of hydroxyurethane A1), by the method described in Example 1. The allophanatisation reaction was started at an NCO content of 43.0% by the addition of 0.5 g of zinc(II)-2-ethyl-1-hexanoate. After reaching an NCO content of 39.9% the reaction mixture was stopped with 1 g of benzoyl chloride and processed as described in Example 1. 662 g of a virtually colourless, clear allophanate polyisocyanate with the following characteristics were obtained:

| | |
|---|---|
| NCO content: | 13.8% |
| Monomeric HDI: | 0.06% |
| Viscosity (23° C.): | 1280 mPas |
| Colour value (APHA): | 22 Hazen |
| NCO functionality: | >3.3 (calculated) |
| Silane group content: | 9.5% (calculated as SiO₃; |
| | mol. weight = 76 g/mol) |

### Example 5 (According to the Invention)

2520.0 g (15.0 mol) of HDI were reacted with 186.9 g (0.7 mol) of the silane-group-containing hydroxyurethane A2) and 27.0 g (0.3 mol) of 1,3-butanediol, corresponding to an amount of 14.4 wt. % relative to the amount of hydroxyurethane A2), by the method described in Example 1. The allophanatisation reaction was started at an NCO content of 44.1% by the addition of 0.5 g of zinc(II)-2-ethyl-1-hexanoate. After reaching an NCO content of 41.0% the reaction mixture was stopped with 1 g of benzoyl chloride and processed as described in Example 1. 667 g of a virtually colourless, clear allophanate polyisocyanate with the following characteristics were obtained:

| | |
|---|---|
| NCO content: | 16.2% |
| Monomeric HDI: | 0.06% |
| Viscosity (23° C.): | 1660 mPas |
| Colour value (APHA): | 22 Hazen |
| NCO functionality: | >3.3 (calculated) |
| Silane group content: | 8.0% (calculated as SiO₃; |
| | mol. weight = 76 g/mol) |

### Example 6 (According to the Invention)

2520.0 g (15.0 mol) of HDI were reacted with 226.1 g (0.7 mol) of the silane-group-containing hydroxyurethane A3) and 27.0 g (0.3 mol) of 1,3-butanediol, corresponding to an amount of 11.9 wt. % relative to the amount of hydroxyurethane A3), by the method described in Example 1. The allophanatisation reaction was started at an NCO content of 43.5% by the addition of 0.5 g of zinc(II)-2-ethyl-1-hexanoate. After reaching an NCO content of 40.4% the reaction mixture was stopped with 1 g of benzoyl chloride and processed as described in Example 1. 718 g of a virtually colourless, clear allophanate polyisocyanate with the following characteristics were obtained:

| | |
|---|---|
| NCO content: | 15.5% |
| Monomeric HDI: | 0.06% |
| Viscosity (23° C.): | 1270 mPas |
| Colour value (APHA): | 23 Hazen |
| NCO functionality: | >3.3 (calculated) |
| Silane group content: | 7.4% (calculated as SiO₃; |
| | mol. weight = 76 g/mol) |

### Example 7 (According to the Invention)

2520.0 g (15.0 mol) of HDI were reacted with 278.1 g (0.9 mol) of the silane-group-containing hydroxyurethane A1) and 64.0 g (0.1 mol) of a linear polycaprolactone polyester with an OH value of 175 mg KOH/g, corresponding to an amount of 35.9 wt. % relative to the amount of hydroxyurethane A1), by the method described in Example 1. The allophanatisation reaction was started at an NCO content of 42.4% by the addition of 0.5 g of zinc(II)-2-ethyl-1-hexanoate. After reaching an NCO content of 39.5% the reaction mixture was stopped with 1 g of benzoyl chloride and processed as described in Example 1. 740 g of a virtually colourless, clear allophanate polyisocyanate with the following characteristics were obtained:

| | |
|---|---|
| NCO content: | 13.8% |
| Monomeric HDI: | 0.04% |
| Viscosity (23° C.): | 1000 mPas |
| Colour value (APHA): | 20 Hazen |
| NCO functionality: | >3.1 (calculated) |
| Silane group content: | 9.2% (calculated as SiO₃; |
| | mol. weight = 76 g/mol) |

### Example 8 (Comparison in Accordance with EP-A 2 014 692)

1680 g (10.0 mol) of hexamethylene diisocyanate (HDI) were reacted with 309 g (1.0 mol) of the silane-group-containing hydroxyurethane A1) by the method described in Example 1. The allophanatisation reaction was started at an NCO content of 40.1% by the addition of 0.5 g of zinc(II)-2-ethyl-1-hexanoate. After reaching an NCO content of 35.9% the reaction mixture was stopped with 1 g of benzoyl chloride and processed as described in Example 1. 789 g of a virtually colourless, clear allophanate polyisocyanate with the following characteristics were obtained:

| | |
|---|---|
| NCO content: | 13.7% |
| Monomeric HDI: | 0.03% |
| Viscosity (23° C.): | 1270 mPas |
| Colour value (APHA): | 21 Hazen |
| NCO functionality: | >3 (calculated) |
| Silane group content: | 9.6% (calculated as SiO₃; |
| | mol. weight = 76 g/mol) |

### Example 9 (Comparison in Accordance with EP-A 2 014 692)

1680 g (10.0 mol) of HDI were reacted with 267 g (1.0 mol) of the silane-group-containing hydroxyurethane A2) by the method described in Example 1. The allophanatisation reaction was started at an NCO content of 41.0% by the addition of 0.5 g of zinc(II)-2-ethyl-1-hexanoate. After reaching an NCO content of 36.7% the reaction mixture was stopped with 1 g of benzoyl chloride and processed as described in Example 1. 690 g of a virtually colourless, clear allophanate polyisocyanate with the following characteristics were obtained:

| | |
|---|---|
| NCO content: | 14.2% |
| Monomeric HDI: | 0.06% |
| Viscosity (23° C.): | 3050 mPas |
| Colour value (APHA): | 19 Hazen |
| NCO functionality: | >3 (calculated) |
| Silane group content: | 11.0% (calculated as SiO₃; |
| | mol. weight = 76 g/mol) |

### Example 10 and 11 (According to the Invention and Comparison)

An amino-functional binder component was prepared from the raw materials listed below in the specified proportions by pre-dispersing for 10 minutes in a high-speed mixer and then grinding in a bead mill whilst cooling:

| | |
|---|---|
| Desmophen NH 1520¹⁾ | 24.32 parts by wt. |
| Desmophen VP LS 2142²⁾ | 7.68 parts by wt. |
| UOP-L powder³⁾ | 3.26 parts by wt. |
| Butyl acetate | 3.34 parts by wt. |
| Bentone 38, 10% digestion⁴⁾ | 3.76 parts by wt. |
| Disperbyk ® 110⁵⁾ | 1.04 parts by wt. |
| Byk ® 085⁵⁾ | 1.14 parts by wt. |
| Chromium oxide green GNM⁶⁾ | 8.29 parts by wt. |
| Bayferrox ® 415⁶⁾ | 1.48 parts by wt. |
| Tronox ® R-KB-4⁷⁾ | 11.88 parts by wt. |
| Barytes EWO⁸⁾ | 31.58 parts by wt. |
| Cab-O-Sil ® TS 720⁹⁾ | 1.51 parts by wt. |
| Tinuvin ® 292¹⁰⁾ | 0.72 parts by wt. |

¹⁾Polyaspartic acid ester, difunctional (delivery form 100%, equivalent weight: 290 g/val NH), Bayer MaterialScience AG, 51368 Leverkusen, Germany
²⁾Blocked cycloaliphatic diamine (delivery form 100%, equivalent weight: 139 g/val NH), Bayer MaterialScience AG, 51368 Leverkusen, Germany
³⁾Molecular sieve, UOP GmbH, 51368 Leverkusen, Germany
⁴⁾Anti-settling agent, Elementis Specialties, 9000 Gent, Belgium
⁵⁾Dispersing additive/venting agent, Byk-Chemie GmbH, 46483 Wesel, Germany
⁶⁾Pigment, Lanxess, 51369 Leverkusen, Germany
⁷⁾Pigment, Tronox Pigments GmbH, 42789 Krefeld, Germany
⁸⁾Filler, Sachtleben Chemie GmbH, 47198 Duisburg, Germany
⁹⁾Rheology additive, Cabot GmbH, 63457 Hanau, Germany
¹⁰⁾Light stabiliser, Ciba, Basle, Switzerland

To produce a ready-to-use coating agent according to the invention, 39.67 parts by weight of the silane-group-containing polyisocyanate according to the invention from Example 1, corresponding to an equivalents ratio of isocyanate groups to isocyanate-reactive groups of 1.1:1, were added to this binder component and mixed in well.

For the purposes of comparison 46.91 parts by weight of the silane-group-containing polyisocyanate from Example 8, likewise corresponding to an equivalents ratio of isocyanate groups to isocyanate-reactive groups of 1.1:1, were added to the same binder component in a second paint batch and likewise mixed in well.

The two paints formulated in this way were applied to a degreased aluminium sheet and to cold-rolled steel using an airless spraying unit, in a wet film thickness of approx. 120 μm in each case, and cured at room temperature (approx. 23° C.) and a relative humidity of approximately 50%.

The pot life of the paint batches was approximately 2 hours in both cases. Table 1 shows the results of the paint tests.

TABLE 1
Drying (in accordance with DIN 53 150) and bond strength
(cross-hatch adhesion tests in accordance with DIN EN ISO 2409)

| | Example 10 | |
|---|---|---|
| | (according to the | Example 11 |
| | invention) | (comparison) |
| Drying times (in accordance with | | |
| DIN 53 150) | | |
| T1 = | 1 h 05 min | 1 h 25 min |
| T6 = | 2 h 35 min | 4 h 10 min |
| Bond strength on aluminium | | |
| after 12 h | GT 0 | GT 0 |
| after 14 d exposure to condensation*⁾ | GT 0 | GT 0 |
| Bond strength on cold-rolled steel | | |
| after 12 h | GT 0 | GT 0 |
| after 14 d exposure to condensation*⁾ | GT 0 | GT 0 |

*⁾in accordance with DIN EN ISO 6270

The comparison shows that the paint produced using the silane-group-containing polyisocyanate crosslinker according to the invention from Example 1 (Example 10) has much shorter drying times in comparison to the paint crosslinked with the silane-group-containing polyisocyanate from Example 8 in accordance with EP-A 2 014 692 (Example 11) with equally excellent adhesion.

1. A method for producing polyisocyanates comprising allophanate groups comprising reacting
A) at least one silane-group-comprising hydroxyurethane and/or hydroxyamide obtained by the reaction of an aminosilane with a cyclic carbonate or lactone, and
B) at least one further polyvalent hydroxy-functional component having a molecular weight in the range of from 62 to 2000 g/mol,
with an amount in molar excess relative to the NCO-reactive groups of components A) and B) of
C) at least one diisocyanate having aliphatically, cycloaliphatically, araliphatically, and/or aromatically bonded isocyanate groups,
and optionally subsequently removing the unreacted diisocyanate excess.

2. The method of claim 1, wherein component A) is the reaction product of an aminosilane of general formula (I) 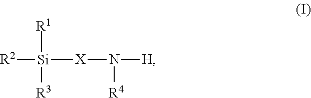
wherein
R¹, R², and R³ are, identically or differently, a saturated or unsaturated, linear or branched, aliphatic or cycloaliphatic radical having up to 18 carbon atoms or an optionally substituted aromatic or araliphatic radical having up to 18 carbon atoms, wherein said saturated or unsaturated, linear or branched, aliphatic or cycloaliphatic radical having up to 18 carbon atoms said optionally substituted aromatic or araliphatic radical can optionally comprise up to 3 heteroatoms selected from the group consisting of oxygen, sulfur, and nitrogen,
X is a linear or branched organic radical comprising at least 2 carbon atoms, which optionally comprise up to 2 imino groups (—NH—), and
R⁴ is hydrogen, a saturated or unsaturated, linear or branched, aliphatic or cycloaliphatic radical having up to 18 carbon atoms, an optionally substituted aromatic or araliphatic radical having up to 18 carbon atoms, or a radical of formula 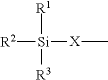
wherein R¹, R², R³, and X are as defined above,
with cyclic carbonates and/or lactones.

3. The method of claim 1, wherein component A) is the reaction product of an aminosilane of general formula (I) 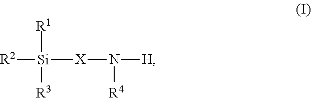
wherein
R¹, R², and R³ are, identically or differently, a saturated, linear or branched, aliphatic or cycloaliphatic radical having up to 6 carbon atoms, and which optionally comprises up to 3 oxygen atoms,
X is a linear or branched alkylene radical having from 2 to 10 carbon atoms, and which optionally comprises up to 2 imino groups (—NH—), and
R⁴ is hydrogen, a saturated, linear or branched, aliphatic or cycloaliphatic radical having up to 6 carbon atoms or a radical of formula 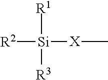
wherein R¹, R², R³, and X are as defined above,
with cyclic carbonates and/or lactones.

4. The method of claim 1, wherein component A) is the reaction product of aminosilane of general formula (I) 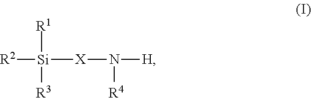
wherein
R¹, R² and R³ are each alkyl radicals having up to 6 carbon atoms and/or alkoxy radicals comprising up to 3 oxygen atoms, with the proviso that at least one of the radicals R¹, R², and R³ is an alkoxy radical,
X is a linear or branched alkylene radical having 3 or 4 carbon atoms, and
R⁴ is hydrogen, a methyl radical, or a radical of formula 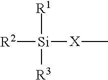
wherein R¹, R², R³, and X have the meaning given above,
with cyclic carbonates and/or lactones.

5. The method of claim 1, wherein component A) is the reaction product of an aminosilane with ethylene carbonate, propylene carbonate, β-propiolactone, γ-butyrolactone, γ-valerolactone, γ-caprolactone, and/or ε-caprolactone.

6. The method of claim 1, wherein component B) is a polyhydric alcohol having a molecular weight in the range of from 62 to 400 g/mol and having 2 to 14 carbon atoms and/or an ester and/or an ether alcohol having a molecular weight in the range of from 106 to 400.

7. The method of claim 1, wherein component B) is a diol and/or triol having having 2 to 6 carbon atoms.

8. The method of claim 7, wherein the total amount of component B) is in the range of from 1 to 70 weight %, relative to the total amount of component A) used.

9. The method of claim 1, wherein component C) is a diisocyanate having aliphatically and/or cycloaliphatically bonded isocyanate groups.

10. The method of claim 1, wherein component C) is a 1,6-diisocyanatohexane, 1-isocyanato-3,3,5-trimethyl-5-isocyanatomethyl cyclohexane, 2,4′- and/or 4,4′-diisocyanatodicyclohexylmethane, or mixtures thereof.

11. The method of claim 1, wherein said reaction is performed in the presence of a catalyst which accelerates the formation of allophanate groups.

12. The method of claim 12, wherein said catalyst is a zinc compound and/or a zirconium compound.

13. An allophanate-group-containing polyisocyanate obtained by the method of claim 1.

14. The allophanate-group-containing polyisocyanate of claim 13, wherein said allophanate-group-containing polyisocyanate is blocked with blocking agents.

15. A polyurethane plastic prepared from the allophanate-group-containing polyisocyanate of claim 13.

16. A coating agent comprising the allophanate-group-containing polyisocyanate of claim 13.

17. A substrate coated with the coating agent of claim 16.
